(19) United States
(12) Patent Application Publication
(10) Pub. No.: US 2005/0284483 A1
(43) Pub. Date: Dec. 29, 2005

(54) Endotracheal tube with integral heart, lung, and temperature monitor

(76) Inventor: Vinu Patel, Dayton, OH (US)

Correspondence Address: WOODARD, EMHARDT, MORIARTY, MCNETT & HENRY LLP, BANK ONE CENTER/TOWER, 111 MONUMENT CIRCLE, SUITE 3700, INDIANAPOLIS, IN, 46204-5137, US

(21) Appl. No.: 11/166,616

(22) Filed: Jun. 24, 2005

Related U.S. Application Data
(63) Continuation-in-part of application No. 10/876,305, filed on Jun. 24, 2004.

(51) Int. Cl. A62B 9/06; A61M 16/00

(52) U.S. Cl. 128/207.14; 128/207.15

(57) ABSTRACT

An endotracheal tube enabling the monitoring of body conditions is described. The endotracheal tube includes a conduit to allow the inflation and deflation of a cuff and, in some embodiments, simultaneously provide a channel to monitor the auditory conditions of a patient such as the heart or lungs. In addition, the endotracheal tube includes a temperature sensing member to measure the temperature of the patient. The temperature, pressure, and auditory aspects of the endotracheal tube are combined into one conduit, in some embodiments. A temperature sensor is placed in the interior of the cuff, in some embodiments, to provide a temperature reading from the air passing in and out of the lungs and from the surrounding tracheal wall. The conduit may be clamped and opened to assist the physician with ease of preparation and insertion of the device into the trachea.

### CROSS REFERENCE TO RELATED APPLICATIONS

This application is a continuation-in-part of prior U.S. patent application Ser. No. 10/876,305 filed Jun. 24, 2004, (attorney docket No. 31147-2) which is incorporated by reference herein in its entirety.

### TECHNICAL FIELD

The present invention relates to endotracheal tubes and more particularly, but not exclusively, to an endotracheal tube that enables the monitoring of patient temperature and heart and lung sounds.

### BACKGROUND

Endotracheal tubes are generally used during anesthesia and also to assist patients with breathing or lung protection. An endotracheal tube facilitates the use of positive pressure ventilation when assistance with breathing is required. For positive pressure ventilation to work, there must be a direct connection to the lungs from the ventilation machine, therefore, an endotracheal tube is required. Endotracheal tubes can also prevent entry of foreign materials into the lungs.

Endotracheal tubes commonly have a cuff, which is an egg-shaped bulb at one end of the tube. The cuff provides a snug attachment to the tracheal wall. The opposite end of the tube exits the patient's mouth or nasal cavity and is hooked to a ventilator. The ventilator pumps air, oxygen and/or anesthesia into the lungs.

Many endotracheal tubes have an inflatable cuff. The inside of the cuff is sometimes filled with a resilient material and most of the time with air. Resilient materials provide a snug fit without causing any damage to the tracheal wall. In addition, many endotracheal tubes include a temperature and/or auditory instrument to assist with the monitoring of body conditions. The temperature of the patient is commonly measured using a temperature sensor placed on the outside or in the outer wall of the endotracheal tube. In addition, many endotracheal tubes have used an inner cuff and an outer cuff to provide audio feedback concerning the patient's heart and lung sounds.

Heretofore, separate tubes have been used for altering pressure within the cuff or monitoring auditory sounds. Alternatively, one tube has been used with separate connectors, one for the device to inflate or deflate the cuff and the other for monitoring the auditory data provided through the tube. One problem with having dual connectors is the awkwardness for the user. In addition, manufacturing costs are increased by the need to provide two different channels. Placement of the temperature sensor on the outside or within the outer section of the tube is costly and difficult. Furthermore, placing the temperature sensor on the outside of the tube exposes the temperature sensor to potential damage from the mishandling of the tube. By placing the sensor on the outside of the tube, the sensor may end up positioned in the air space between the tracheal wall and the tube. The sensor's effectiveness is decreased when the sensor is in the air space because of poor heat transfer.

Therefore, needs remain in this area of technology.

### SUMMARY

One aspect includes a method for determining body conditions during medical treatment. The method includes the steps of providing an endotracheal tube for insertion into a trachea including a cuff containing resilient material, a temperature sensor, a stop cock; and a conduit operatively coupled to a universal connector; preparing the tube for insertion into the trachea; inserting the tube into the trachea; preparing the tube for monitoring body conditions; and using the endotracheal tube for monitoring body conditions.

Another aspect includes an endotracheal tube. The endotracheal tube includes an elongated member including a proximal portion and a distal portion, the elongated member further including an outer and inner surface defining a lumen therethrough; a cuff including a flexible outer cover operatively coupled to the outer surface of the distal portion of the elongated member and an adhesive joint defining the cuff into two separate areas including a proximal cuff area defined between the flexible outer cover and the adhesive joint, and a distal cuff area defined between the flexible outer cover and the adhesive joint; a first conduit integrally formed with the elongated member having a proximal end and a distal end, wherein the distal end of the conduit lies in said proximal cuff area and the proximal end of the conduit lies outside of the lumen; a second conduit integrally formed with the elongated member having a proximal end and a distal end, wherein the distal end of the second conduit lies inside said distal cuff area and the proximal end of the conduit lies outside of the lumen.

Yet a further aspect is an endotracheal tube. The endotracheal tube includes an elongated member including a proximal portion and a distal portion, the elongated member further including an outer and inner surface defining a lumen therethrough; a cuff including a flexible inner cover and a flexible outer cover operatively coupled to the outer surface of the distal portion of the elongated member, the cuff including an inner cuff area defined by the flexible inner cover containing a resilient material, and an outer cuff area defined by the flexible outer cover containing air, wherein said inner cuff area is enclosed within said outer cuff area; a first conduit integrally formed with the elongated member having a proximal end and a distal end, wherein the distal end of the conduit lies in said inner cuff area and the proximal end of the conduit lies outside of the lumen; a second conduit integrally formed with the elongated member having a proximal end and a distal end, wherein the distal end of the second conduit lies inside said outer cuff area and the proximal end of the conduit lies outside of the lumen; and a temperature sensing member including a temperature sensor, wherein the temperature sensor lies inside the inner cuff area.

Another aspect is an endotracheal tube including an elongated member including a proximal portion and a distal portion, the elongated member further including an outer and inner surface defining a lumen therethrough; a cuff including a flexible inner cover and a flexible outer cover operatively coupled to the outer surface of the distal portion of the elongated member, the cuff including an inner cuff area defined by the flexible inner cover containing air and an outer cuff area defined by the flexible outer cover containing a resilient material, wherein the inner cuff area is enclosed within the outer cuff area; a first conduit integrally formed with the elongated member having a proximal end and a distal end, wherein the distal end of the conduit lies in the inner cuff area and the proximal end of the conduit lies outside of the lumen; a second conduit integrally formed with the elongated member having a proximal end and a distal end, wherein the distal end of the second conduit lies inside the outer cuff area and the proximal end of the conduit lies outside of the lumen; and a temperature sensing member including a temperature sensor, wherein the temperature sensor is coupled to the outer surface of the elongated member

### BRIEF DESCRIPTION OF THE DRAWINGS

FIG. 1 illustrates an endotracheal tube according to one embodiment of the present invention.

FIG. 2 is an enlarged view of the cross-section of the middle of the endotracheal tube illustrated in FIG. 1.

FIG. 3 is a cross-section through the cuff of the endotracheal tube illustrated in FIG. 1.

FIG. 4 is a schematic diagram illustrating the procedure for intubating a patient using the endotracheal tube illustrated in FIG. 1.

FIG. 5 illustrates an alternate embodiment of an endotracheal tube for children.

FIG. 6 illustrates another alternate embodiment of an endotracheal tube.

FIG. 7 illustrates yet another alternate embodiment of an endotracheal tube.

FIG. 8 illustrates a further embodiment of an endotracheal tube.

### DETAILED DESCRIPTION OF SELECTED EMBODIMENTS

For the purposes of promoting an understanding of the principles of the claimed methods and endotracheal tubes, reference will now be made to the embodiments illustrated in the drawings and specific language will be used to describe the same. It will nevertheless be understood that no limitation of the scope of the invention, as defined by the claims, is thereby intended. Any alterations, modifications, and further applications of the principles of the methods and endotracheal tubes as illustrated are contemplated as would normally occur to one skilled in this art.

An endotracheal tube has a deflatable cuff containing a resilient material, such as foam. A conduit has one end that lies in the area inside of the deflatable cuff. The other end is attached to a universal connector for attaching to a device for either applying or removing pressure from the inside of the cuff or attaching an auditory sensing device. The conduit also contains a temperature sensing device including a temperature sensor that lies within the interior of the cuff. The temperature sensing device may be connected to a temperature monitor in one embodiment. The endotracheal tube allows the monitoring of the heart and lungs sounds through the auditory signals received through the conduit and also measures the body temperature through the temperature sensing device. In addition, the temperature sensing device passes through the conduit so that all three functions of deflation, temperature measurement, and auditory monitoring are combined through one conduit. Furthermore, efficient temperature sensing is enabled as well as simplicity in manufacturing. Some alternate embodiments are illustrated that provide variations of the original design.

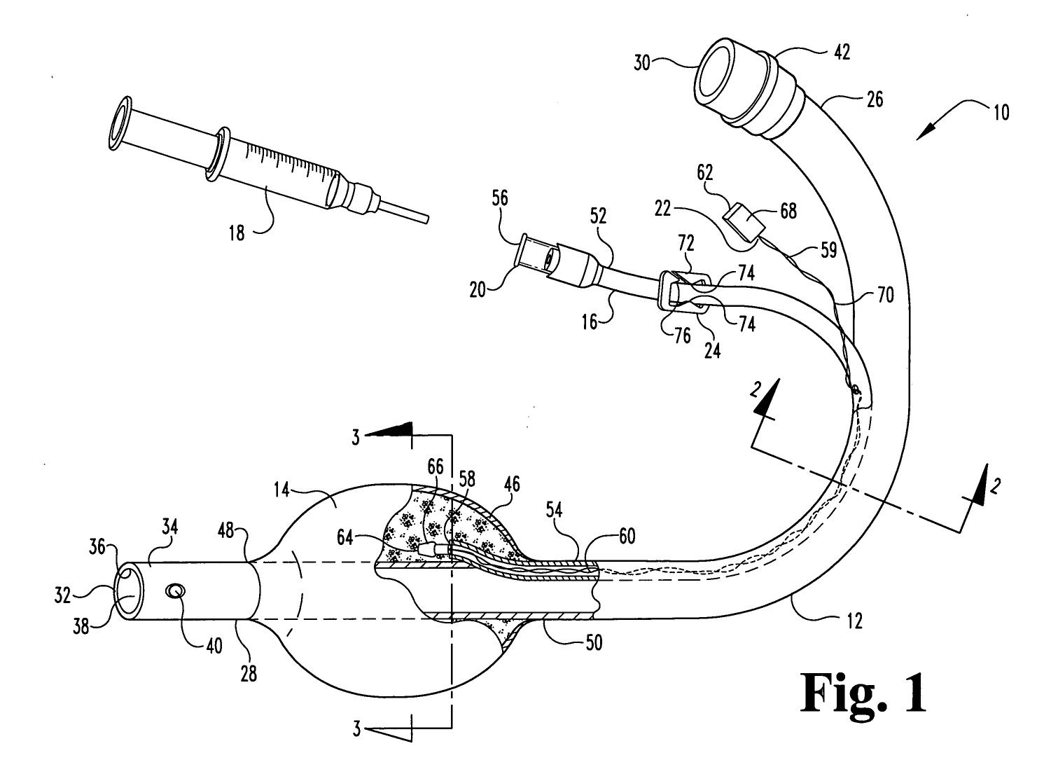

Referring now to the figures, FIG. 1 illustrates endotracheal tube 10. The endotracheal tube 10 includes elongated member 12 to provide fluid communication between the ventilator and the patient's lungs. Endotracheal tube 10 also has cuff 14 to provide a snug fixing point between the endotracheal tube 10 and the patient's tracheal wall (not shown). The endotracheal tube 10 also includes a conduit 16 that enables pressure to be removed from the inside of the cuff 14 by pressure altering device 18. In addition, conduit 16 is operatively coupled to a universal connector 20. The universal connector 20 is constructed and arranged so it can be connected to the pressure altering device 18 or to an auditory sensing device, such as a stethoscope. Endotracheal tube 10 also includes a temperature sensing member 22 for measuring the temperature of the patient. Finally, the endotracheal tube 10 includes a clamp 24 for either restricting or allowing air to flow through the conduit 16.

In the illustrated embodiment, the elongated member 12 is a flexible tube. In other embodiments, the elongated member 12 is a rigid curved tube. The elongated member 12 may be composed of any biocompatible material. Some materials include, but are not limited to, polyvinylchloride, latex, silicone, rubber, or other materials readily apparent to those skilled in the art. The elongated member 12 is curved to conform to the shape of the human trachea. In differing embodiments, the elongated tube 12 passes through the nasal passage or alternatively through the throat. Commonly, the elongated member 12 is composed of a clear material to aid in viewing any obstructions that may become embedded therein. In some embodiments, however, it is contemplated that markings are placed upon the elongated member 12 or it is composed of a hazy or even opaque material. Polyvinylchloride materials are generally preferred, because a number of humans are allergic to latex materials. The elongated member 12 has a proximal portion 26 and a distal portion 28. The distal portion 28 will lie inside of the trachea of the patient after insertion of the tube into the trachea. The proximal portion 26 exits either the nasal cavity or the throat of the patient. The elongated member 12 also has a proximal end 30 and a distal end 32. The proximal end 30 is also located outside of the body after insertion of the tube into the trachea. The distal end 32 provides fluid communication with the lungs of the patient when inserted into the trachea. The elongated member 12 also includes an outer surface 34 and an inner surface 36. The combination of the outer surface 34 and the inner surface 36 defines a lumen 38 therein. The lumen 38 is generally uniform throughout the length of the endotracheal tube 10 and allows fluid communication between the ventilator (not shown) and the lungs of the patient. The illustrated embodiment has a lumen 38 that has a uniform diameter throughout the length of the elongated member 12. Other embodiments contemplate having a lumen 38 that has varying diameters throughout the length of the elongated member 12. The distal portion 28 also defines a vent 40. The vent 40 assists in supplying the anesthetic gases, such as nitrogen dioxide, air, and/or oxygen to pulmonary system of the patient. The elongated member 12 also includes a connector 42 that is operatively coupled to the proximal portion 26 of the elongated member 12. The connector 42 connects the endotracheal tube 10 to a ventilator or other anesthesia machine (not shown). The connector 42 may be bonded to the elongated member 12 through the use of an adhesive, an interference fit, heat sealing, or any other method readily apparent to one skilled in the art. In one alternative embodiment, the elongated member 12 is a one-piece integral member with the connector 42. Other embodiments contemplate a connector that may be inserted or removed from the elongated member 12. Moreover, other embodiments contemplate using a system of connectors to connect the endotracheal tube 10 to a ventilation machine (not shown) or an anesthesia machine (not shown).

The endotracheal tube 10 also includes a cuff 14. The cuff 14 includes a flexible cover 46 that deforms upon the removal of pressure inside of the cuff 14. In addition, the flexible cover 46 defines the outer limits of the cuff 14. In the illustrated embodiment, the cuff 14 is filled with a resilient foam material. Upon the creation of a vacuum inside the cuff 14, the foam material will collapse. Once atmospheric pressure is reintroduced inside of the cuff 14, the foam will expand back out to its earlier shape. In the illustrated embodiment, the cuff 14 defines an egg-like shape. It is contemplated that other embodiments have a cuff that defines an alternate shape that is suitable to provide snug fixation against the trachea. As FIG. 1 illustrates, the elongated member 12 passes through the center of the cuff 14. This enables fluid communication between the ventilator or anesthesia machine and the patient's lungs. The cuff 14 is operatively coupled to the elongated member 12 at a first seal 48 and a second seal 50. The seals 48, 50 are made by any method readily apparent to one skilled in the art. One example, among others, seals the flexible cover 46 using a biocompatible adhesive. Another example would be a heat seal. The method of sealing should hermetically seal the flexible cover 46 to the elongated member 12. The flexible cover 46 may be composed of any biocompatible material. Polyvinylchloride is one preferred material because of its hypoallergenic qualities. In addition, silicone that is flexible and deformable is another preferred material.

The endotracheal tube 10 also includes a conduit 16. The conduit 16 is integrally formed with the elongated member 12 as illustrated in FIG. 1. In one embodiment, the conduit 16 is a flexible tube throughout its entire length. In another embodiment, the conduit 16 is a flexible tube for the portion of the tube that is outside of the elongated member 12. The remainder of the portion of the conduit 16 is a bore through the side wall of the elongated member 12 defined by the outer surface 34 and the inner surface 36. The conduit 16 provides fluid communication to and from the inside of the cuff 14. Conduit 16 may be formed of any flexible material. For one example, polyvinylchloride is a suitable material. Other materials commonly known to those skilled in the art may also be used. The conduit 16 includes a proximal portion 52 and a distal portion 54. The proximal portion 52 lies outside of the elongated member 12. The distal portion 54 lies inside the lumen 38 of the elongated member 12 or directly within the wall of the elongated member 12 defined by the outer surface 34 and the inner surface 36. The conduit 16 also includes a proximal end 56 and a distal end 58. The proximal end 56 will connect to a pressure altering device or an auditory sensing device. The distal end 58 lies inside of the inner chamber of the cuff 14. The distal end 56 of the conduit 16 is the universal connector 20. The universal connector 20 is operatively coupled to the conduit 16. The universal connector 20 is constructed and arranged to connect to a pressure altering device, such as the pressure altering device 18 in the illustrated embodiment, or other types of pressure altering devices.

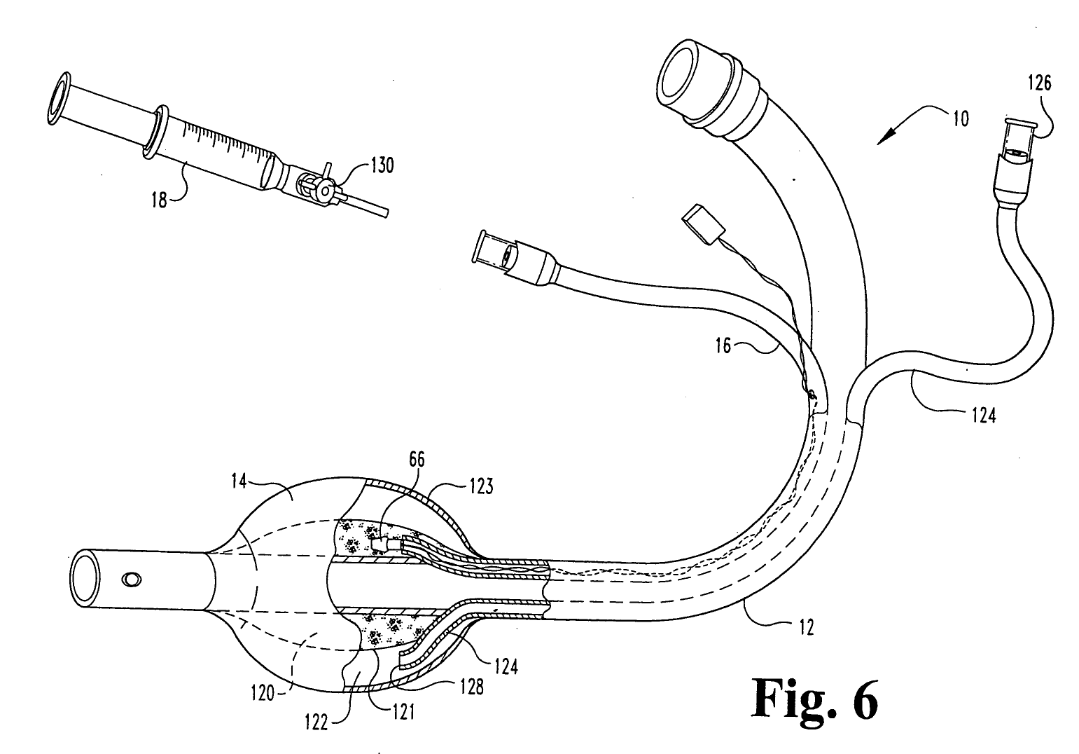

For example, pressure altering device 18 as illustrated is a syringe with a nozzle. Other types of pressuring altering devices apparent to those skilled in the art are contemplated. For instance, the pressure altering device 18 can be a syringe with a nozzle and a stop cock 130 as illustrated in FIG. 6. Moreover, the stop cock 130 does not have to be coupled to the pressure altering device 18, it can be coupled to the conduit 16 in alternate embodiments. The universal connector 20 also can connect to an auditory sensing device. A common auditory sensing device would be a stethoscope. Other types of auditory sensing devices readily apparent to those skilled in the art are used in alternate embodiments.

The endotracheal tube 10 also includes a temperature sensing member 22. The temperature sensing member 22 of the illustrated embodiment is a thermocouple. Other temperature sensing members 22 readily apparent to those skilled in the art are also contemplated. The temperature sensing member 22 has a proximal portion 59 and a distal portion 60. The distal portion 60 lies either in the distal portion 54 of the conduit 16 as shown in FIG. 1, or lies within the wall defined by the elongated member 12, or in the lumen 38 of the elongated member 12. The proximal portion 59 of the temperature sensing member 22 lies outside of the elongated member 12. The temperature sensing member 22 also includes a proximal end 62 and a distal end 64. The distal end 64 of the temperature sensing member 22 is a temperature sensor 66. The temperature sensor 66 converts the amount of heat absorbed by the temperature sensor 66 into an electrical signal that is passed through the electrical conduit 70 to the electrical connector 68. The electrical connector 68 is connected to a temperature monitoring device (not shown). In the illustrated embodiment, the electrical connector 68 is a dual-holed connector composed of plastic. The holes of the electrical connector 68 are sized to receive prongs from the electrical monitoring device. Other embodiments contemplate other electrical connectors for connecting to a temperature monitoring device readily apparent to those skilled in the art. In the illustrated embodiment, the temperature sensor 66 lies in the inner chamber directly inside the cuff 14. The temperature sensor 66 picks up heat in order to provide data concerning the temperature of the patient. The temperature sensing capabilities are disclosed hereinbelow with reference to FIG. 3.

The endotracheal tube 10 also includes a clamp 24. In the illustrated embodiment, the clamp 24 includes a push face 72, pinchers 74 and a catch ridge 76. Other embodiments, however, use a different form of clamp 24. For one example, in one alternate embodiment the clamp 24 is a stop cock, affixed to the conduit 16 or the pressure altering device 18, that can be turned to prevent fluid communication through the conduit 16. In another embodiment, the endotracheal tube 10 does not use a clamp 24 at all and the physician has to manually hold the conduit shut. Other embodiments use other forms of a clamp 24 that are readily apparent to those skilled in the art.

The clamp 24, of the illustrated embodiment, is used to prevent and allow fluid communication through the conduit 16. For example, when a vacuum is being created inside of the cuff 14 using the pressure altering device 18, after the vacuum is created, the clamp 24 may be closed. Closing the clamp 24 prevents the cuff 14 from expanding as the pressure returns to the normal atmospheric pressure. Alternatively, the clamp 24 may be used to prevent the treating physician from hearing auditory signals if desired. The clamp 24 is operated by the user pressing down on the push face 72 which causes the clamp 24 to bend and catch on the catch ridge 76, thereby causing the pinchers 74 to close off fluid communication through the conduit 16. In the illustrated embodiment, the clamp 24 is composed of plastic. Other materials readily apparent to those skilled in the art may be used.

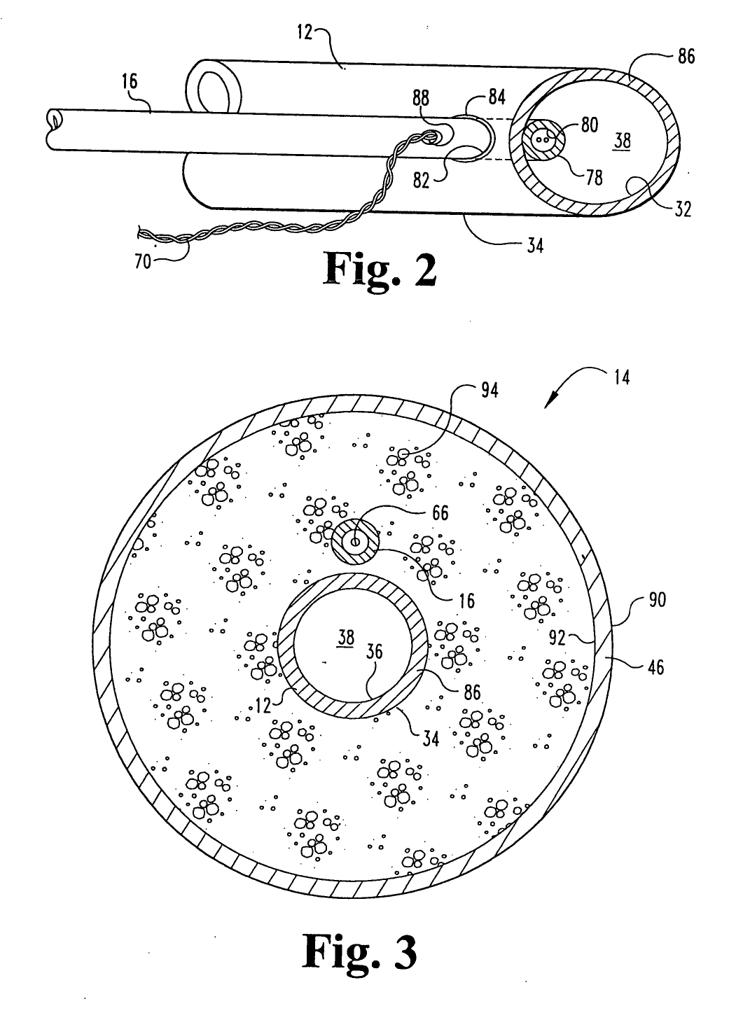

FIG. 2 illustrates the conduit 16 and the temperature sensing member 22 moving from inside the elongated member 12 to outside of the elongated member 12. The conduit 16 passes through the lumen 38 and is affixed to the inside surface 32 of the elongated member 12 in the embodiment illustrated in FIG. 2. The conduit 16 has an outer surface 78 and an inner surface 80. The electrical conduit 70 passes through the center of the conduit 16 defined by the outer surface 78 and the inner surface 80. The conduit 16 then passes through an aperture 82 that is defined in the side of the elongated member 12. The aperture 82 and the conduit 16 are hermetically sealed using a biocompatible adhesive, a heat seal, or others methods of sealing commonly known to those skilled in the art. In an alternate embodiment, the conduit 16 passes through the inner wall 86 of elongated member 12. In that embodiment, there is no need to seal the conduit 16 and the elongated member 12. The conduit 16 also includes a small aperture 88 to allow the electrical conduit 70 to pass outside of the conduit 16. The small aperture 88 and the electrical conduit 70 must be hermetically sealed to allow proper pressure transfer and auditory readings to occur through the conduit 16. In an alternate embodiment, the electrical conduit 70 and temperature sensing member 22 remain completely inside of the conduit 16 and conduit 16 has a double connector end. The double connector end has the electrical connector 68 and the universal connector 20 side-by-side, so that aperture 88 does not have to be formed into conduit 16. Another embodiment locates the aperture 88 closer to the universal connector 20. The illustrated embodiment allows for the temperature sensing member 22 and the conduit 16 to be spatially separated from one another to assist with the connection to the sensing equipment.

FIG. 3 illustrates a cross-section of the cuff 14. The cuff 14 has the flexible cover 46, an outer surface 90 and an inner surface 92. The cuff 14 also defines an inner chamber 94 between flexible cover 46 and the outer surface 34 of the elongated member 12. In the illustrated embodiment, the inner chamber is filled with resilient material. Some materials include, but are not limited to foam, sponge, or other resilient materials. The cuff 14 operates by creating a vacuum using the pressure altering device 18 in the inner chamber 94 causing the resilient material to compress. The flexible cover 46 also shrinks around the resilient material on the inner chamber 94. After the endotracheal tube 10 is inserted into the trachea, the pressure inside of the inner chamber 94 returns to atmospheric. The resilient material expands slowly and gently to provide a snug attachment to the tracheal wall without causing any tracheal damage. In addition, the conduit 16 remains in the inner chamber 94 of the cuff 14.

Auditory signals from the lungs and heart are received into the conduit 16. For example, the expanding and contracting of the lungs produces fluid flow through the lumen 38, the sound is transferred through the wall 86 and into the foam in the inner chamber 94. The sound is then continually transferred into the center of the conduit 16 through the conduit 16 and up to the universal connector 20. Therefore, the sounds of the expansion and contraction of the lungs may be monitored by the physician. Similarly, the heart will produce a sound throughout the entire medium portion of the body. The sound will eventually run into the flexible cover 46 and will be transferred through the flexible cover 46 into the inner chamber 94 to the conduit 16. The foam helps to transfer the sound waves. Sound travels quicker through solid material than air. Thus, the physician may also monitor the heart of the patient.

In addition, heat from the air transferred through the lumen 38 and from the outer body through the trachea wall and the flexible cover 46 will be passed through the inner chamber 94 to the temperature sensor 66. This configuration provides a balanced temperature reading that incorporates both temperature reading from the air passing into and out of the lungs and from the surrounding tracheal wall. This provides an accurate temperature reading that can be assessed by the sensor 66 and identified by the temperature monitoring device that is connected to the temperature sensing member 22. Therefore, the physician can monitor the activities of the lungs, heart and temperature simultaneously. This is critical during anesthesiology because many patients may react negatively to the anesthesia gas or to other variables that can cause serious injury or even death.

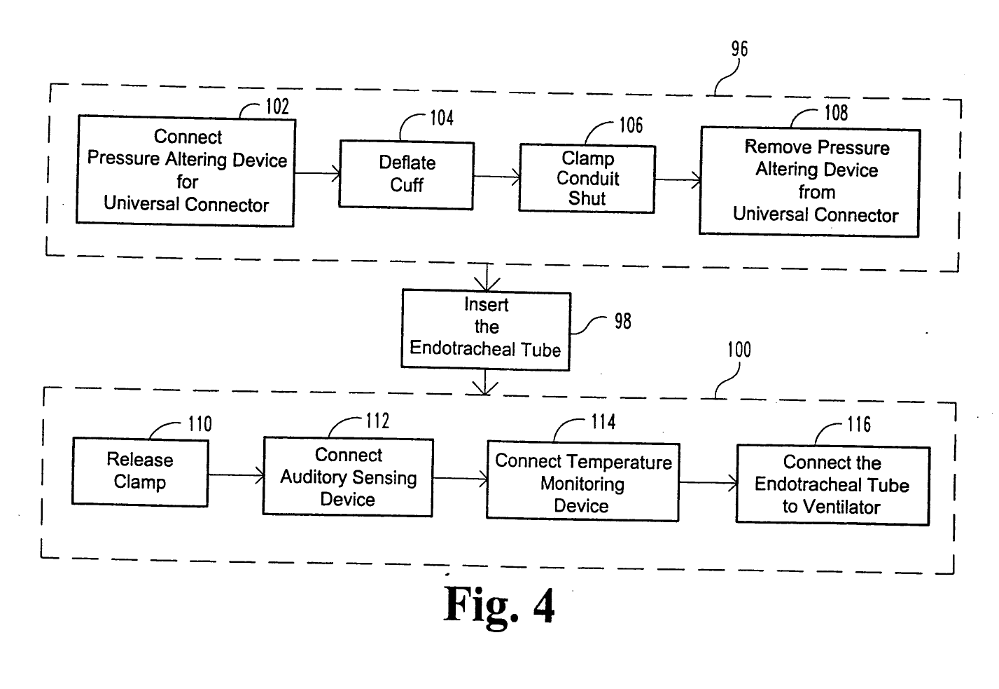

FIG. 4 illustrates the procedure for operating the device. In broad terms, the procedure for using the endotracheal tube 10 is to first prepare the endotracheal tube 10 for insertion into the trachea during an initial preparation step 96. Next, the endotracheal tube 10 is inserted into the trachea during the insertion step 98. Finally, the endotracheal tube 10 is prepared for monitoring body conditions during the final preparation step 100. FIG. 4 illustrates that the initial preparation step 96 and the final preparation step 100 consists of substeps 102 through 116 to fully prepare the patient before, during and after intubation.

The endotracheal tube 10 is prepared for insertion during the initial preparation step 96. The initial preparation step 96 consists of a first substep 102. During the substep 102, the pressure altering device 18, such as the pressure altering device 18 illustrated in FIG. 1, is connected to the universal connector 20 of the endotracheal tube 10. In the substep 104, the cuff 14 is deflated using the pressure altering device 18. For example, the pressure altering device 18 illustrated in FIG. 1 is a syringe. The syringe is inserted into the universal connector 20 and air is drawn out of the cuff 14 through the conduit 16 by pulling backwards on the end of the syringe. Therefore, a vacuum is created in the cuff 14 causing the foam or resilient material inside to collapse and shrink the cuff 14. By doing so, the cuff 14 is now of a suitable size to enter the mouth or the nasal passage and go into the trachea without becoming stuck in the trachea or damaging the trachea. Before this can happen, however, a substep 106 must be performed. In the substep 106, the conduit 16 is clamped shut using the clamp 24. The clamp may be the clamp 24 illustrated in FIG. 1 or any other type of clamp suitable to completely close the conduit 16, such as the stop cock 130 illustrated in FIG. 6. By clamping the conduit 16 shut, fluid communication between the universal connector 20 and the inside of the cuff 14 is prohibited. By doing so, the vacuum is maintained inside of the cuff 14 and the cuff 14 remains deflated. Only now may the physician proceed to a substep 108. The substep 108 is to remove the pressure altering device 18 from the universal connector 20 so that the endotracheal tube 10 may be inserted into the nasal or throat passage. The cuff 14 continues to be deflated because of the clamp 24 while the physician prepares to insert the endotracheal tube 10 into the trachea.

The second and most important general step is the insertion of the endotracheal tube 10 into the trachea. This is performed during an insertion step 98. The physician may need a stylus to help insert a endotracheal tube 10 that is formed of a flexible material. In an alternate embodiment where the endotracheal tube 10 is composed of a rigid material, the stylus may not be necessary. Many times the endotracheal tube 10 contains markings along the side to assist the physician with determining the proper length of the tube to be inserted into the trachea. The markings also provide clear and accurate viewing during this sensitive procedure. Once the endotracheal tube 10 is properly positioned inside of the trachea, the physician can proceed to the second preparation step 100.

In the second preparation step 100, the physician ensures that the endotracheal tube 10 is snuggly affixed to the trachea and that all connections are made for proper monitoring and care of the patient. After the endotracheal tube 10 has been properly positioned, a substep 110 may be performed. The substep 110 is to release the clamp 24 or stop cock 130 to reopen the conduit 16. By doing do, the physician restores atmospheric pressure to the inner chamber 94 of the cuff 14. This causes the resilient material inside of the inner chamber 94 to expand in a slow and gradual manner. This allows the cuff 14 to conform itself to the shape of the trachea without causing any trauma or damage to the trachea. Therefore, a snug fixation is achieved between the cuff 14 and the tracheal wall of the patient. Once the endotracheal tube 10 is snuggly affixed, the physician may proceed to a substep 112. The substep 112 occurs when the physician connects an auditory sensing device to the universal connector 20. The auditory sensing device in one embodiment is a stethoscope (not shown). Other embodiments contemplate other types of auditory sensing devices. Upon connection of the auditory sensing device, the physician may monitor the sounds produced by the lungs, heart, or both. These sounds may be extremely helpful to the physician while monitoring the body conditions of the patient during anesthesia or other procedures. Next, the physician can move to a substep 114 and connect the temperature monitoring device. The temperature monitoring device may connect to a machine that reads out an analog or digital reading of the temperature of the patient. Commonly, the temperature monitoring device includes an alarm that sounds when the temperature of the patient rises above or below an acceptable level. Knowing the temperature of the patient can be critical to the health and the safety of the patient during anesthesia or other procedures.

Next, the physician moves to a substep 116 and connects the endotracheal tube 10 to the ventilator. The ventilator may provide air, oxygen, or anesthesia depending on the needs of the procedure. The ventilator provides positive pressure to the lungs to inflate them and reduces pressure to deflate the lungs in order to keep the patient breathing during a procedure. The substeps 112, 114 and 116 may be varied in accordance with the preference of the physician. Similarly, the substep 112 may follow the substep 110. The substeps 114 and 116 may also proceed either the substeps 110 or 112.

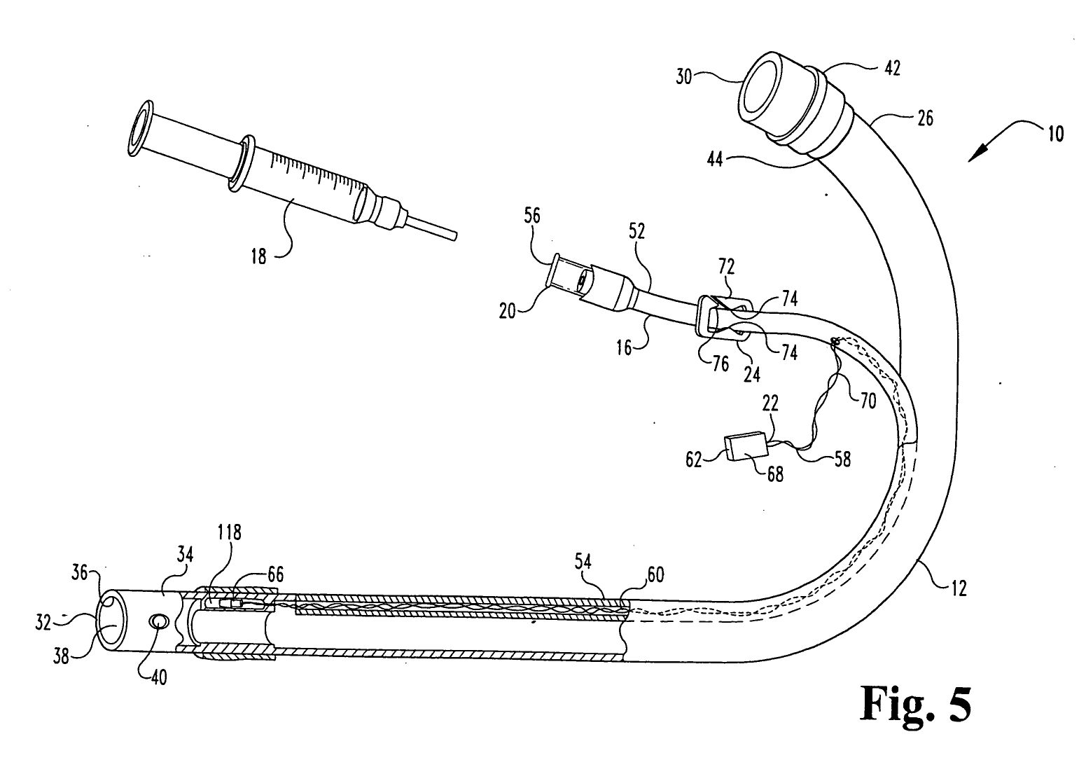

Referring now to FIG. 5, an alternate embodiment of the endotracheal tube 10 is illustrated. The illustrated endotracheal tube 10 is intended to be used with children six years old and under. As with the embodiment illustrated in FIG. 1, the conduit 16 enables the physician to listen to heart and lungs sounds, however, the endotracheal tube 10 does not include a cuff that can be inflated or deflated. The endotracheal tube 10 defines dead space 118 at the end of the conduit 16 to assist in receiving auditory signals from the heart and lungs. The endotracheal tube 10 also includes a temperature sensing member 22 for sensing the temperature of the patient. Like the embodiment illustrated in FIG. 1, the temperature sensing member 22 runs through the inside of the conduit 16. The temperature sensor 66 lies in the dead space 118 defined by the endotracheal tube 10. Positioning the temperature sensor 66 in the dead space 118 assists with obtaining an accurate temperature. The small size of the trachea of a child prohibits the use of a cuff similar to the one illustrated in FIG. 1. As another variation on this embodiment, the circumferential area of the outer surface of the endotracheal tube surround the dead space 118 can be covered with a foam sleeve. Covering the section of the tube over the dead space 118 with a foam sleeve assists in preventing damage to the child's trachea. In alternative embodiments, a larger portion of the tube, or even the entire section of the tube that will lie in the trachea is covered with a foam sleeve to prevent damage to the trachea.

Referring now to FIG. 6, an alternate embodiment of endotracheal tube 10 is illustrated. The endotracheal tube 10 includes a cuff 14 having an inner cuff area 120 and an outer cuff area 122. The inner cuff area 120 is defined by a flexible inner cover 121 filled with a resilient material, such as foam, and the temperature sensor 66 lies inside of the inner cuff area 120. The inner cuff area 120 is deflated by drawing out air through the conduit 16. The outer cuff area 122 is defined by outer flexible cover 123 and is filled with air. A second conduit 124 includes a proximal end 126 and a distal end 128. The distal end 128 is located inside of the outer cuff area 122 and allows the air inside of the outer cuff area 122 to be removed. In addition, the outer cuff area 122 can be inflated by using a pressure altering device 18. The pressure altering device 18 illustrated includes a stop cock 130. The stop cock 130 is used to allow or prevent air from exiting pressure altering device 18. In addition, it is appreciated that stop cock 130 can be directly coupled to conduit 16.

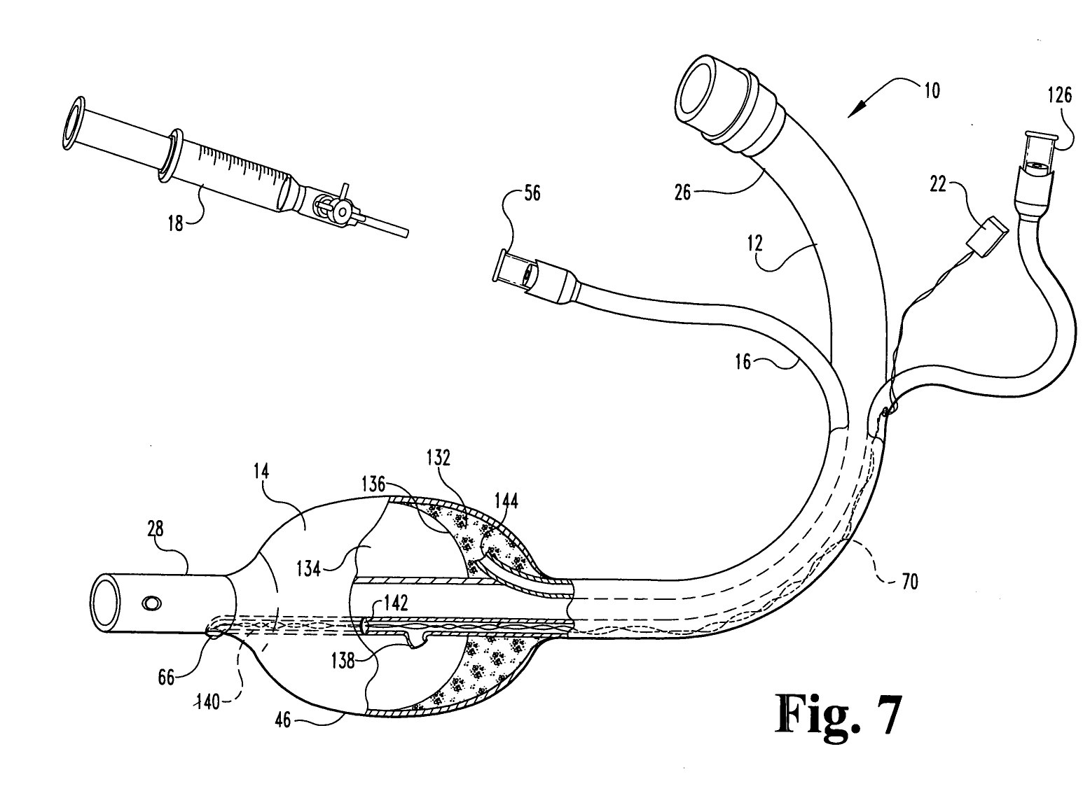

Referring now to FIG. 7, yet another alternate embodiment of an endotracheal tube 10 is illustrated. The endotracheal tube 10 includes a cuff 14 that is separated into a proximal cuff area 132 and a distal cuff area 134. Unlike the embodiment in FIG. 6, that contains one inner cuff area 120 and one outer cuff area 122, the cuff areas 132 and 134 do not lie completely inside or around one another. The proximal cuff area 132 is filled with a resilient material to enable a controlled expansion of the cuff 14 and to provide structure to the cuff 14. The distal cuff area 134 is empty and inflatable. The combination of the air-filled distal cuff area 134 and resilient material filled proximal cuff area 132 improves the transfer of sound. The proximal cuff area 132 and the distal cuff area 134 are separated by an adhesive joint 136. In most embodiments, the adhesive joint 136 is created by fixing the flexible outer cover 46 of the proximal cuff area 132 to the flexible outer cover 46 of the distal cuff area 134. The flexible outer cover 46 of the proximal and distal cuff areas 132, 134 can be fixed using a biocompatible adhesive, heat sealing, or other method readily apparent to those skilled in the art. The second conduit 126 includes an inflating section 138 that allows fluid communication through the second conduit 126 enabling the distal cuff area 134 to inflate. The inflating section 138 is separated from an extending section 140 by a closure 142. The closure 142 can be any biocompatible item allowing the electrical conduit 70 to continue along the second conduit 126 into the extending section 140 but preventing any air from flowing through. A drop of adhesive, plastic, or the like are some examples of a closure 142. The extending section 140 enables the temperature sensor 66 to rest outside of the cuff 14. The first conduit 16 has an auditory section 144 that can pick up sounds and transfer them to an auditory device, such as a stethoscope, that connects to the first conduit 16.

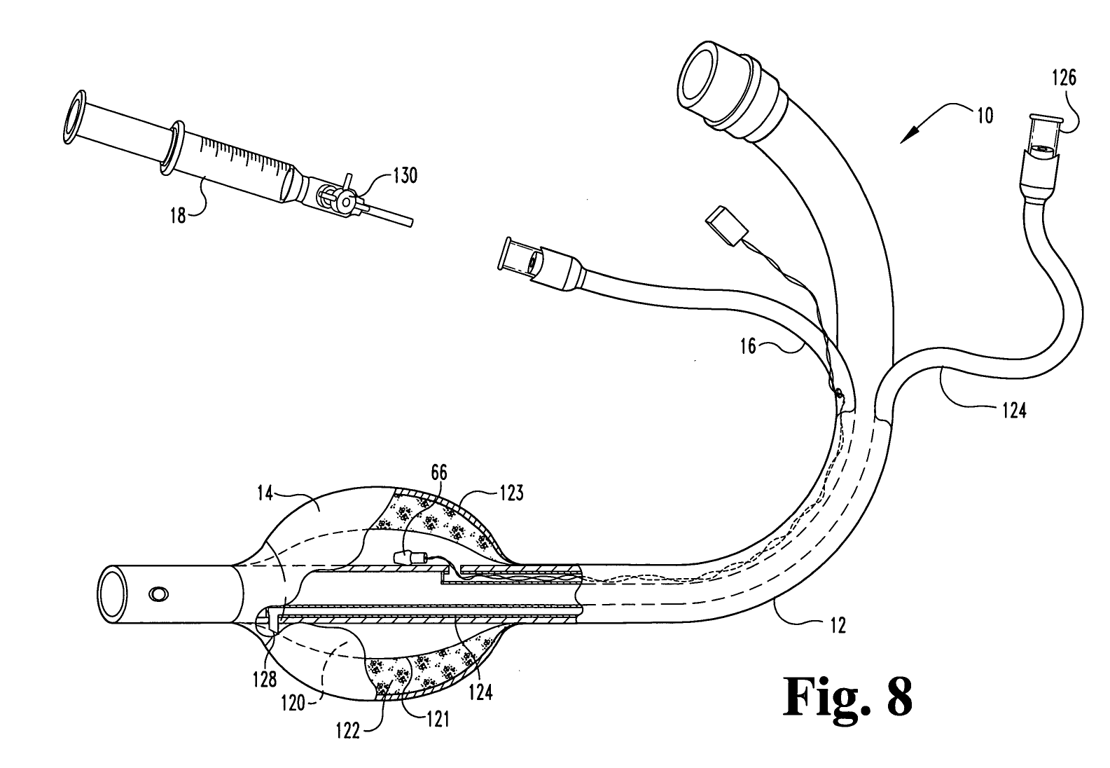

An additional embodiment is disclosed in FIG. 8. The device utilizes a two cuff design, such as in FIG. 6, but alters the structure. Primarily, the outer cuff area 122 is filled with a resilient material. The outer cuff area 122 can be deflated using the second conduit 124. The inner cuff area 120 is inflated and deflated by moving air through the conduit 16. The temperature sensor 66 is placed on the portion of the outside of the elongated member 12 that is enclosed by the inner cuff area 120. The resilient material of the outer cuff area 122 helps to transfer the sounds to an attachable auditory sensing device.

While the invention has been illustrated and described in detail in the drawings and foregoing description, the same is to be considered as illustrative and not restrictive in character, it being understood that only the preferred embodiment has been shown and described and that all changes and modifications that come within the spirit of the invention are desired to be protected.

1. A method for determining body conditions during medical treatment comprising the steps of:
(a) providing an endotracheal tube for insertion into a trachea including a cuff containing resilient material, a temperature sensor, a stop cock; and a conduit operatively coupled to an universal connector;
(b) preparing the tube for insertion into the trachea;
(c) inserting the tube into the trachea;
(d) preparing the tube for monitoring body conditions;
(e) using the endotracheal tube for monitoring body conditions.

2. The method of claim 1, wherein preparing the endotracheal tube for insertion into the trachea further comprises the steps of:
(b1) connecting a pressure altering device to the universal connector;
(b2) deflating the cuff;
(b3) preventing fluid communication with the cuff using the stop cock; and
(b4) removing the pressure altering device from the universal connector.

3. The method of claim 1, wherein preparing the endotracheal tube for monitoring body conditions further comprises the steps of:
(d1) resuming fluid communication by opening the stop cock;
(d2) connecting an auditory sensing device to the universal connector;
(d3) connecting a temperature monitoring device to the temperature sensor; and
(d4) connecting the endotracheal tube to a ventilator.

4. An endotracheal tube comprising:
an elongated member including a proximal portion and a distal portion, the elongated member further including an outer and inner surface defining a lumen therethrough;
a cuff including a flexible outer cover operatively coupled to the outer surface of the distal portion of the elongated member and an adhesive joint defining the cuff into two separate areas including a proximal cuff area defined between the flexible outer cover and the adhesive joint, and a distal cuff area defined between the flexible outer cover and the adhesive joint;
a first conduit integrally formed with the elongated member having a proximal end and a distal end, wherein the distal end of the conduit lies in said proximal cuff area and the proximal end of the conduit lies outside of the lumen;
a second conduit integrally formed with the elongated member having a proximal end and a distal end, wherein the distal end of the second conduit lies inside said distal cuff area and the proximal end of the conduit lies outside of the lumen.

5. The endotracheal tube of claim 4, wherein said proximal cuff area contains a resilient material.

6. The endotracheal tube of claim 4, wherein said proximal cuff area contains air.

7. The endotracheal tube of claim 4, wherein said distal cuff area contains a resilient material.

8. The endotracheal tube of claim 4, wherein said distal cuff area contains air.

9. The endotracheal tube of claim 4, further comprising a universal connector operatively coupled to the proximal end of at least one of said conduits constructed and arranged to connect to an auditory sensor and a pressure altering device.

10. The endotracheal tube of claim 4, further including a temperature sensing member having a temperature sensor, wherein the temperature sensor lies outside the cuff proximal to the flexible outer cover.

11. The endotracheal tube of claim 4, wherein the flexible covers are composed of polyvinylchloride.

12. The endotracheal tube of claim 4, further including a closure, wherein said closure prevents fluid communication to an extending portion of said second conduit.

13. The endotracheal tube of claim 4, wherein the resilient material is foam.

14. The endotracheal tube of claim 6, wherein the temperature sensing member is a thermocouple.

15. An endotracheal tube comprising:
an elongated member including a proximal portion and a distal portion, the elongated member further including an outer and inner surface defining a lumen therethrough;
a cuff including a flexible inner cover and a flexible outer cover operatively coupled to the outer surface of the distal portion of the elongated member, the cuff including an inner cuff area defined by the flexible inner cover containing a resilient material and an outer cuff area defined by the flexible outer cover containing air, wherein said inner cuff area is enclosed within said outer cuff area;
a first conduit integrally formed with the elongated member having a proximal end and a distal end, wherein the distal end of the conduit lies in said inner cuff area and the proximal end of the conduit lies outside of the lumen;
a second conduit integrally formed with the elongated member having a proximal end and a distal end, wherein the distal end of the second conduit lies inside said outer cuff area and the proximal end of the conduit lies outside of the lumen; and
a temperature sensing member including a temperature sensor, wherein the temperature sensor lies inside the inner cuff area.

16. The endotracheal tube of claim 15, further including an universal connector operatively coupled to the proximal end of at least one of said conduits constructed and arranged to connect to an auditory sensor and a pressure altering device.

17. The endotracheal tube of claim 15, wherein said conduits are flexible tubes.

18. The endotracheal tube of claim 15, wherein the conduit is a flexible tube for the portion of the conduit outside of the elongated body, and a bore through the elongated body for the portion of the conduit integrally formed within the elongated body.

19. The endotracheal tube of claim 15, wherein the flexible covers are composed of polyvinylchloride.

20. The endotracheal tube of claim 15, wherein the elongated member is formed of a flexible material.

21. The endotracheal tube of claim 15, the elongated member further including a proximal end and a distal end and a connector operatively coupled to the proximal end.

22. The endotracheal tube of claim 15, further including at least one stop cock to control the amount of fluid communication passing through at least one of the conduits.

23. The endotracheal tube of claim 15, wherein the resilient material is foam.

24. The endotracheal tube of claim 15, wherein the temperature sensing member is a thermocouple.

25. An endotracheal tube comprising:
an elongated member including a proximal portion and a distal portion, the elongated member further including an outer and inner surface defining a lumen therethrough;
a cuff including a flexible inner cover and a flexible outer cover operatively coupled to the outer surface of the distal portion of the elongated member, the cuff including an inner cuff area defined by the flexible inner cover containing air and an outer cuff area defined by the flexible outer cover containing a resilient material, wherein said inner cuff area is enclosed within said outer cuff area;
a first conduit integrally formed with the elongated member having a proximal end and a distal end, wherein the distal end of the conduit lies in said inner cuff area and the proximal end of the conduit lies outside of the lumen;
a second conduit integrally formed with the elongated member having a proximal end and a distal end, wherein the distal end of the second conduit lies inside said outer cuff area and the proximal end of the conduit lies outside of the lumen; and
a temperature sensing member including a temperature sensor, wherein the temperature sensor is coupled to the outer surface of the elongated member.

26. The endotracheal tube of claim 25, further including an universal connector operatively coupled to the proximal end of at least one of said conduits constructed and arranged to connect to an auditory sensor and a pressure altering device.

27. The endotracheal tube of claim 25, wherein said conduits are flexible tubes.

28. The endotracheal tube of claim 25, wherein the conduit is a flexible tube for the portion of the conduit outside of the elongated body, and a bore through the elongated body for the portion of the conduit integrally formed within the elongated body.

29. The endotracheal tube of claim 25, wherein the flexible covers are composed of polyvinylchloride.

30. The endotracheal tube of claim 25, wherein the elongated member is formed of a flexible material.

31. The endotracheal tube of claim 25, the elongated member further including a proximal end and a distal end and a connector operatively coupled to the proximal end.

32. The endotracheal tube of claim 25, further including at least one stop cock to control the amount of fluid communication passing through at least one of the conduits.

33. The endotracheal tube of claim 25, wherein the resilient material is foam.

34. The endotracheal tube of claim 25, wherein the temperature sensing member is a thermocouple
